(19) United States
(12) Patent Application Publication
(10) Pub. No.: US 2024/0091111 A1
(43) Pub. Date: Mar. 21, 2024

(54) METHODS OF MASKING OFF-TASTES

(71) Applicant: Givaudan SA, VERNIER (CH)

(72) Inventors: Norikazu SAJI, Chartham, Canterbury Kent (GB); Julie CHARPENTIER, Basel (CH)

(21) Appl. No.: 18/275,080

(22) PCT Filed: Feb. 24, 2022
(86) PCT No.: PCT/EP2022/054603

§ 371 Date: Jul. 31, 2023

(30) Foreign Application Priority Data
Feb. 25, 2021 (GB) 2102675.2

(51) Int. Cl. A61K 8/21; A61K 8/27; A61Q 11/00

(52) U.S. Cl. CPC A61K 8/21; A61K 8/27; A61Q 11/00; A61K 2800/74

(57) ABSTRACT

Disclosed is a method of reducing off-notes, in particular bitter off-taste and/or astringent off-tastes of oral care products.

### TECHNICAL FIELD

The disclosure relates to a method of reducing off-notes, in particular bitter off-taste and/or astringent off-tastes of oral care products. The disclosure further relates to reduced bitter and/or astringency oral care products, and to the use of certain compounds to reduce bitterness and/or astringency in oral care products.

### BACKGROUND

Oral care products available on the market do contain quite often active ingredients possessing a bitter and/or astringent off-taste. Said active ingredients may be of diverse origin, examples of which include dextromethorphan, guaifenesin, caffeine, doxylamine, sildenafil citrate, loperamide and also zinc salts. Of particular concern, however, is the use of stannous fluoride (SnF₂), which despite its benefits in oral hygiene, may cause unpleasant tastes and may leave a gritty feeling in the mouth.

Materials causing an undesired off-taste are, for example, cetyl pyridinium chloride, chlorhexidine, certain surfactants (e.g. cocamidopropyl betaine (CAS 61789-40-0), which is for example available under the trade name Tego® Betain from Evonik), aluminum salts (such as aluminum lactate), zinc salts (such as zinc citrate, zinc sulfate, and zinc oxide), and potassium salts (such as potassium citrate and potassium nitrate).

Whereas there are benefits to add ingredients, such as stannous fluoride and/or zinc salts, to oral care products, it is desired to reduce and or mask their undesired off-taste.

It is known in the art that the addition of sweeteners, such as sodium saccharin and/or sucralose, can at least partially reduce these undesired bitter and/or astringent off-taste. Nevertheless, there remains a need to provide compounds that are useful for reducing and/or masking an undesired off-taste (such as bitter and/or astringency).

### SUMMARY

In accordance with a first aspect of the present invention there is provided a method of masking or reducing bitter and/or astringency taste of a composition to be received orally (e.g. oral care product), comprising the addition to said composition of an effective amount of 2′-hydroxypropiophenone.

In accordance with a second aspect of the present invention there is provided an oral care composition comprising an ingredient providing a bitter and/or astringent off-taste, and 2′-HOPP, optionally in combination with methyl-fluor-hydroxybenzoate.

In certain embodiments the bitter off-taste ingredient is selected from the group consisting of stannous fluoride, cetyl pyridinium chloride, chlorhexidine, and cocamidopropyl betaine.

In certain embodiments the astringent off-taste ingredient is selected from the group consisting of zinc salts, potassium salts and aluminum salts.

### DETAILED DESCRIPTION

The present invention is based on the surprising finding that 2′-hydroxypropiophenone (CAS 610-99-1; 2′-HOPP) has excellent bitter and astringency masking properties.

2′-hydroxypropiophenone is known in the art as a replacement for methyl salicylate (2-hydroxybenzoic acid methyl ester).

PCT Publication WO98/31242 describes the use of 2′-hydroxypropiophenone as a flavorant to enhance the flavour of wintergreen-flavored products and to contribute a wintergreen note to non-wintergreen-flavored products, thus being suitable to partially or completely replacing methyl salicylate.

PCT Publication WO2001/068044 describes oral compositions comprising a wintergreen flavor imparting ingredient and having an alkaline pH, wherein the wintergreen flavor imparting ingredient is 2′-hydroxypropiophenone.

However, no evidence can be found in the literature that 2′-hydroxypropiophenone when combined with bitter tasting or astringent tasting ingredients results in a composition which is perceived less bitter and/or less astringent.

Thus, there is provided herein in a first aspect a method of reducing of a bitterness and/or astringency of a composition to be received orally (e.g. oral care product), comprising the addition to said composition an effective amount of 2′-hydroxypropiophenone.

Depending on the source of 2′-hydroxy propriophenone, the commercially available quality may contain traces of 4-chloro propiophenone, phenyl propionate and/or phenol. Said compounds possess a very strong, cresolic, chemical and animalic odor profile which may overturn the positive effects of 2′-hydroxy propriophenone. Accordingly, preferably the commercially available 2′-hydroxy propriophenone may be distillated before used in oral care compositions.

In one particular embodiment, the commercial 2′-hydroxy propriophenone is distilled bulb-to bulb at about 120° C. and 0.075 mbar to essentially remove 4-chloro propiophenone and phenol. By “essentially” is meant that the final quality of 2′-hydroxy propriophenone contains less than 0.5 weight % of 4-chloro propiophenone and less than 0.5 weight % of phenol. Instead of distillation other means well known to the skilled person may be used to remove the impurities.

The applicant has found that if 2′-hydroxypropiophenone is used in combination with methyl-fluor-hydroxybenzoate (e.g. methyl 4-fluoro-2-hydroxybenzoate), even better results can be achieved in terms of reducing bitterness and astringency of an oral care product.

Thus there is provided in a further embodiment a method of reducing of a bitterness and/or astringency of a composition to be received orally (e.g. oral care product), comprising the addition to said composition an effective amount of 2′-hydroxypropiophenone, and optionally methyl-fluoro-hydroxybenzoate (e.g. methyl 4-fluoro-2-hydroxybenzoate, and methyl 2-fluoro-6-hydroxybenzoate).

In one particular embodiment, the orally received composition comprises as a bitter inducing compound stannous fluoride (SnF₂).

Stannous fluoride (SnF₂) is a common active ingredient in toothpaste and has been recognized by the American Dental Association (ADA) as an effective agent in reducing tooth decay. The difference between stannous fluoride and other fluoride types is that it includes key benefits such as:
Anti-cavity: While all fluoride types have been proven to help prevent and control cavities by strengthening tooth enamel and dentin, stannous fluoride has been shown to make dental surfaces more resistant to bacterial acids.
Antimicrobial: Stannous fluoride is also recognized for its antimicrobial properties, meaning that it can kill the bacteria found in plaque that cause gum disease and tooth decay.
Anti-hypersensitivity: Stannous fluoride helps reduce teeth sensitivity resulting from hot, cold, and sweet foods and beverages, it does this forming a layer over open dentinal tubules (tiny channels in the dentin that lead directly to the sensitive pulp at the tooth's center).

Stannous fluoride, when present in oral care products, such as toothpaste, is present at concentrations of about 0.2 to 0.6 weight % based on the oral care product. Such products are often perceived possessing quinine like bitter taste with a hint of astringent taste.

In another particular embodiment, the orally received composition comprises as an astringent inducing compound a zinc salts (including zinc oxide, zinc chloride, zinc sulfate, and zinc lactate).

Toothpaste with zinc seems to have great inhibitory efficacy on dentine demineralization, thus zinc in form of, e.g., zinc oxide, is quiet often added to oral care products.

Zinc oxide, when present in oral care products, such as toothpaste, is present at concentrations of 0.1 to 0.6 weight % based on the oral care product. Such products are often perceived possessing a strong astringent taste.

Astringency, in respect to compositions to be received orally, is defined as a complex group of sensations experienced when a substance causes the oral surfaces to feel rough, the mouth to feel dry, and the mucosal surfaces to tighten, draw or pucker.

Bitter is one out of five taste sensations, which are detected by taste receptors in the taste buds located on the surface of the tongue and other areas of the mouth and throat. Bitter substances known to the layman are caffeine and quinine.

The term “oral care product” as used herein refers to non-food compositions that are designed to be taken into the mouth to deliver a variety of benefits. Such compositions include dentifrices, mouthwashes, mouth sprays and gargle compositions, breath strips (edible films placed in the oral cavity to administer thereto an active agent such as a flavourant or breath-freshening agent), and chewing gums. The term “dentifrice”, as used herein, means toothpaste, oral care gels or liquids, unless otherwise specified. The dentifrice composition may be a single-phase composition or it may be a combination of two or more separate dentifrice compositions. The dentifrice composition may be in any desired form, such as deep striped, surface striped, multilayered, having the gel surrounding the paste, or any combination thereof.

By “effective amount of 2′-hydroxypropiophenone” is meant an amount which is sufficient to noticeably reduce the undesired off-taste.

As a skilled person may acknowledge, the amount to be sufficient for the reduction of the off-taste depends on several parameters. Firstly, the compound causing the undesired off-taste and secondly, the amount present in the oral care product. Accordingly, it is not easy to provide generally valid quantities. However, a skilled person will easily be able to evaluate the required amounts by routine experimentation.

As one specific embodiment one may mention an oral care product comprising stannous fluoride at concentration of about 0.2 to 0.6 weight %. To noticeable reduce the bitter off-taste of such a product, the addition of about at least 0.05 weight % (e.g. about 0.075-0.2, including 0.1 weight %) of 2′-hydroxypropiophenone, optionally further comprising about 0.00001-0.001 (e.g. 0.001) weight % of methyl-fluoro-hydroxybenzoate, is sufficient.

As another specific embodiment one may mention an oral care product comprising cetyl pyridinium chloride (which is known for its antibacterial properties) at concentration of about 0.01 to 0.1 weight % (e.g. 0.05, 0.06, 0.07, 0.08 weight %). To noticeable reduce the bitter off-taste of such a product, the addition of about at least 0.05 weight % (e.g. about 0.075-0.2, including 0.1 weight %) of 2′-hydroxypropiophenone, optionally further comprising about 0.00001-0.001 (e.g. 0.001) weight % of methyl-fluoro-hydroxybenzoate, is sufficient.

As a further embodiment one may mention an oral care product comprising zinc salt (e.g. zinc oxide, zinc citrate, zinc chloride, zinc sulfate, and/or zinc lactate) at a concentration of about 0.5 to 5 weight % (e.g., from 0.1 to 2.0 weight %, which includes 0.5 to 1.0 weight %). To noticeable reduce the astringent off-taste of such a product, the addition of about at least 0.05 weight % (e.g. about 0.075-0.2, including 0.1 weight %) of 2′-hydroxypropiophenone, optionally further comprising about 0.00001-0.001 (e.g. 0.001) weight % of methyl-fluoro-hydroxybenzoate, is sufficient.

The reduced bitter and/or astringency oral care product of the present invention may also comprise one or more additional ingredients or excipients conventionally used in conjunction with oral care products for example, additional flavour compounds (other than 2′-HOPP) and other auxiliary agents commonly used in the art.

Examples of known additional flavour ingredients may be found in one of the FEMA (Flavour and Extracts Manufacturers Association of the United States) publications or a compilation thereof which is available from and published by FEMA and contains all FEMA GRAS (Generally Regarded As Safe) publications from 1965 to present, eg GRAS 21 published 2003, or in Allured's Flavor and Fragrance Materials 2004, published by Allured Publishing Inc.

Examples of other auxiliary agents commonly used in the art include, for example, surfactants, emulsifiers, solvents, colorants, preservatives, antioxidants, antimicrobial agents, enzymes, vegetal or mineral oils, fats, proteins, solubilisers, sugar derivatives, vitamins, polyols including sorbitol, organic acids, artificial sweeteners, polymers, thickeners, chewing gum bases, and oral malodour counteracting actives. Some oral care products contain alcohols, in particular lower alcohols (C₁-C₄).

Examples of known auxiliary agents for oral care products may be found in Gaffar, Abdul, Advanced Technology, Corporate Technology, Department of Oral Care, Colgate-Palmolive Company, Piscataway, NJ, USA. Editor(s): Barel, Andre O.; Paye, Marc; Maibach, Howard I., Handbook of Cosmetic Science and Technology (2001), p. 619-643. Publisher: Marcel Dekker, Inc., New York, N. Y, and in Cosmetics: Science and technology, 2nd edition, p. 423-563. Edited by M. S. Balsam and E. Sagarin, Wiley Interscience, 1972.

The invention is now further described with reference to the following non-limiting examples. These examples are for the purpose of illustration only and it is understood that variations and modifications can be made by one skilled in the art.

### Example 1: Purification of 2′-Hydroxypropiophenone

A sample of 2′-hydroxypropiophenone (10.00 g, 66.6 mmol, odor description: cresolic, animalic, wintergreen) obtained from Xinchem Corporation was distilled bulb-to-bulb at 120° C./0.075 mbar to yield sensorily purified 2′-hydroxy propiophenone (9.90 g, 6.59 mmol, odor description: wintergreen). The thus obtained 2′-hydroxy propiophenone quality contained less than 0.3 weight % of chloro propiophenone.

### Example 2: Toothpaste with Stannous Fluoride

To an unflavored toothpaste base free of sweeteners comprising 0.454 weight stannous fluoride (i.e. 0.16 weight % fluoride ions), of
a) 0.1 weight % of 2′-HOPP
b) 0.09 weight % of 2′-HOPP and 0.01 weight % of methyl 4-fluoro-2-hydroxybenzoate @ 1% in propylene glycol

A piece of the thus-prepared toothpaste was put on a toothbrush and a panelist's teeth were brushed.

The unflavored toothpaste base was described to possess quinine like bitter taste with a hint of astringent taste. It was observed that the bitterness stays for a few minutes in the mouth.

Composition a) in comparison to the unflavored toothpaste base was assessed to possess less bitterness and astringent taste. On top the overall taste impression was improved bringing a pleasant phenolic aromatic character.

Composition b) in comparison to the unflavored toothpaste base was assessed to cover the bitterness and astringent taste extremely well. On top the addition of the mixture b) provides a rounded sweet sugarly note.

On a linear scale from 1-10 (1: not bitter/not astringent; 10: very bitter/astringent), the toothpaste base comprising 0.454 weight % stannous fluoride was rated 7, composition a) was rated 4, and composition b) was rated 3 by the expert panelists.

### Example 3: Toothpaste with Zinc Oxide

To an unflavored chalk based toothpaste free of sweeteners comprising 0.2 weight zinc oxide, of
a) 0.1 weight % of 2′-HOPP
b) 0.09 weight % of 2′-HOPP and 0.01 weight % of methyl 4-fluoro-2-hydroxybenzoate @ 1% in propylene glycol

A piece of the thus-prepared toothpaste was put on a toothbrush and a panelist's teeth were brushed.

The unflavored chalk based toothpaste was described to possess a chalky dry taste with strong astringent taste. It was observed that the astringent taste stays for a few minutes in the mouth.

Composition a) in comparison to the unflavored chalk based toothpaste was perceived less astringent and more pleasant in taste.

Composition b) in comparison to composition b) was perceived even less astringent providing a smooth mouth feel.

1. A method of reducing an off-taste of a composition to be received orally, comprising adding to said composition 2′-HOPP and optionally methyl-fluoro-hydroxybenzoate.

2. The method according to claim 1 wherein 2′-HOPP was purified by removing 4-chloro propiophenone to a level of less than 0.5 weight %.

3. The method according to claim 1 wherein 2′-HOPP is essentially free of a compound selected from 4-chloro propiophenone, phenol, and/or phenyl propionate.

4. An oral care composition comprising an ingredient providing a bitter and/or astringent off-taste, and 2′-HOPP.

5. The oral care composition according to claim 4 wherein the bitter off-taste ingredient is selected from cetyl pyrimidinium, cocamidopropyl betaine, and/or zinc salts.

6. The oral care composition according to claim 4 wherein the astringent off-taste ingredient is selected from stannous fluoride, potassium and/or aluminum salts.

7. The oral care composition according to claim 4 wherein the composition additionally comprises methyl-fluoro-hydroxybenzoate.

8. The oral care composition according to claim 4 wherein 2′-HOPP is essentially free of 4-chloro propiophenone.

9. The oral care composition according to claim 4 wherein 2′-HOPP is essentially free of 4-chloro propiophenone, phenol, and/or phenyl propionate.

10. The oral care composition according to claim 9 wherein 2′-HOPP contains less than 0.5 weight % of 4-chloro propiophenone.

11. The method according to claim 1 comprising adding to said composition 2′-HOPP and methyl-fluoro-hydroxybenzoate.

12. The oral care composition according to claim 5, wherein the composition additionally comprises methyl-fluoro-hydroxybenzoate.

13. The oral care composition according to claim 6, wherein the composition additionally comprises methyl-fluoro-hydroxybenzoate.

14. The oral care composition according to claim 5 wherein 2′-HOPP is essentially free of 4-chloro propiophenone.

15. The oral care composition according to claim 6 wherein 2′-HOPP is essentially free of 4-chloro propiophenone.

16. The oral care composition according to claim 7 wherein 2′-HOPP is essentially free of 4-chloro propiophenone.

17. The oral care composition according to claim 5 wherein 2′-HOPP is essentially free of 4-chloro propiophenone, phenol, and/or phenyl propionate.

18. The oral care composition according to claim 6 wherein 2′-HOPP is essentially free of 4-chloro propiophenone, phenol, and/or phenyl propionate.

19. The oral care composition according to claim 7 wherein 2′-HOPP is essentially free of 4-chloro propiophenone, phenol, and/or phenyl propionate.

20. The oral care composition according to claim 8 wherein 2′-HOPP is essentially free of 4-chloro propiophenone, phenol, and/or phenyl propionate.
